(19) United States
(12) Patent Application Publication
(10) Pub. No.: US 2025/0306002 A1
(43) Pub. Date: Oct. 2, 2025

(54) METHOD FOR EVALUATING CITRUS FRUIT CRACKING AND USE THEREOF

(71) Applicant: Zhejiang University, Hangzhou City (CN)

(72) Inventors: Chongde Sun, Hangzhou City (CN); Yue Wang, Hangzhou City (CN); Chen Yunyi, Hangzhou City (CN); Xie Fei, Hangzhou City (CN); Peng Wang, Hangzhou City (CN); Dengliang Wang, Hangzhou City (CN); Yang Xu, Hangzhou City (CN); Jinping Cao, Hangzhou City (CN); Shaojia Li, Hangzhou City (CN)

(73) Assignee: Zhejiang University, Hangzhou City (CN)

(21) Appl. No.: 18/731,610

(22) Filed: Jun. 3, 2024

(30) Foreign Application Priority Data
Mar. 26, 2024 (CN) 202410353809.6

(51) Int. Cl. G01N 33/02

(52) U.S. Cl. CPC G01N 33/025

(57) ABSTRACT

Provided is a method for evaluating citrus fruit cracking. In the method, five indicators for evaluating citrus fruit cracking are determined and subjected to correlation analysis with field experiment data of fruit cracking rates of citrus fruits, and analysis results show that these five indicators are positively correlated with a cracking rate of fruits. By detecting these five indicators, the correlation of fruit cracking characteristics with a variety and cultivation conditions can be accurately described to fundamentally understand a cause for fruit cracking. In addition, it is convenient to determine the five indicators, and the five indicators can be detected in both fruits on a fruit tree and picked fruits. The method provides a technical support for improving the fruit cracking characteristics of a variety through breeding or guiding measures for controlling the fruit cracking including application of a specified fertilizer or reduction of a watering quantity.

### CROSS-REFERENCE TO RELATED APPLICATION

This patent application claims the benefit and priority of Chinese Patent Application 2024103538096 filed with the China National Intellectual Property Administration on Mar. 26, 2024, the disclosure of which is incorporated by reference herein in its entirety as part of the present application.

### TECHNICAL FIELD

The present disclosure belongs to the technical field of evaluation of citrus fruit cracking, and specifically relates to a method for evaluating citrus fruit cracking.

### BACKGROUND

Citrus fruit cracking is a common physiological disease for citrus fruits and usually occurs during the ripening of fruits. Citrus fruit cracking is characterized by a crack in the peel to expose the flesh. The fruit cracking has an adverse impact on quality and market value of the citrus, including the following aspects: (1) Quality loss: the cracked peel makes the appearance of a fruit poor to affect a sales value of the citrus, and consumers generally tend to buy fruits with intact appearance. (2) Susceptibility to pathogens: a crack becomes a channel for the invasion of pathogens, which increases the risk of a fruit to be attacked by pathogens such as fungi and bacteria. (3) Water loss: the cracked peel will cause a fruit to lose water, thereby affecting the taste and storage performance. (4) Poor eating experience: the fruit cracking usually causes the flesh to become uneven, which may affect the edible experience. (5) Reduced yield: if a large number of fruits undergo fruit cracking, the overall yield may be affected.

Currently, the evaluation of citrus fruit cracking is mainly based on field observation, and the citrus fruit cracking is evaluated through a ratio of the number of cracked fruits to the total number of fruits on an individual fruit tree. There is still a lack of methods for quantitative evaluation of individual fruits. Such a rough indicator can hardly reflect the relationship among a variety, a management level, and an environmental factor. For example, fruit cracking rates of some citruses may be as high as 80% and can vary among different years, regions, and orchard management levels, indicating that the fruit cracking is a complicated trait influenced by a variety of factors. The single fruit cracking rate cannot comprehensively evaluate the fruit cracking problem, and an accurate and multi-dimensional description method is required.

The occurrence of citrus fruit cracking is usually related to the following factors: (1) Improper water management: the excessive or insufficient irrigation may cause the citrus fruit cracking. (2) Climatic factors: sudden changes in climatic conditions such as humidity and temperature may trigger the fruit cracking. (3) Nutritional imbalance: the excessive or deficient nutrient supply may also cause the fruit cracking. (4) Pests and diseases: some pests and diseases may cause the peel to crack, thereby providing an opportunity for the invasion of pathogens. However, the citrus fruit cracking is usually a result of a combination of various factors, and thus accurate and quantitative descriptive indicators and methods are required to determine which aspects of fruits are related to the fruit cracking.

### SUMMARY

An objective of the present disclosure is to provide a method for evaluating citrus fruit cracking. The method can both quantitatively describe a quality of a citrus fruit itself and present a correlation relationship with a citrus fruit cracking rate in the field, such that a cause of fruit cracking can be fundamentally understood to guide practical activities.

The present disclosure provides the use of one or more of five indicators in evaluation of citrus fruit cracking,
where the five indicators include peel hardness, fruit turgor pressure, fruit cracking site at fruit turgor pressure limit, injected water volume at fruit turgor pressure limit, and cracked fruit proportion index;
the fruit turgor pressure is a pressure value produced inside a citrus fruit when the citrus fruit cracks due to water injected into the citrus fruit from a central axis of the citrus fruit;
the fruit cracking site at fruit turgor pressure limit is a site of a crack of a citrus fruit, and the fruit cracking site at fruit turgor pressure limit is quantified by the following scoring manner: there is cracking in a peel equatorial plane: 5 points, there is cracking between a fruit equatorial plane and a fruit pedicel or a fruit umbilicus: 4 points, there is cracking around a fruit umbilicus: 3 points, there is cracking at a fruit umbilicus: 2 points, there is cracking at a fruit pedicel: 1 point, there is a wound on the fruit: 0 point, and there is cracking around a needle: 0 point;
the injected water volume at fruit turgor pressure limit is an amount of injected water when a citrus fruit cracks;
a determination method of the cracked fruit proportion index includes: injecting water into fruits from fruit pedicels of the fruits in an amount 5% to 15% of a weight of each fruit, taking a ratio of the number of fruits in which a peel cracks within 24 h to the total number of fruits in which the water is injected as a cracking ratio Cr after the water injection, and calculating the cracked fruit proportion index with the cracking ratio Cr after the water injection, a score S of a peel cracking site, and a time t when a peel cracks according to a predetermined formula:
cracked fruit proportion index=0.347×(25−t)×(1+S)×(1+Cr), where the score S of a peel cracking site is determined by the same scoring manner as the fruit cracking site at fruit turgor pressure limit, and the time t when a peel cracks refers to a tₜₕ h after the water is injected into a fruit.

Preferably, the peel hardness includes a peel hardness of an intact fruit and an isolated peel hardness; and the peel hardness is determined by a texture analyzer with a depth of 3 mm to 8 mm.

Preferably, the texture analyzer has a probe diameter of 2 mm and a travel speed of 1.0 mm/s.

Preferably, the peel hardness is negatively correlated with a fruit cracking rate of citrus fruits.

Preferably, the fruit turgor pressure and the injected water volume at fruit turgor pressure limit each are negatively correlated with the fruit cracking rate of citrus fruits; and the fruit cracking site at fruit turgor pressure limit is positively correlated with a fruit cracking rate of citrus fruits.

Preferably, the cracked fruit proportion index is positively correlated with a fruit cracking rate of citrus fruits.

The present disclosure also provides a method for evaluating citrus fruit cracking, including: evaluating fruit cracking of a citrus fruit by determining one or more of five indicators,
where the five indicators include peel hardness, fruit turgor pressure, fruit cracking site at fruit turgor pressure limit, injected water volume at fruit turgor pressure limit, and cracked fruit proportion index;
the fruit turgor pressure is a pressure value produced inside a citrus fruit when the citrus fruit cracks due to water injected into the citrus fruit from a central axis of the citrus fruit;
the fruit cracking site at fruit turgor pressure limit is a site of a crack of a citrus fruit, and the fruit cracking site at fruit turgor pressure limit is quantified by the following scoring manner: there is cracking in a peel equatorial plane: 5 points, there is cracking between a fruit equatorial plane and a fruit pedicel or a fruit umbilicus: 4 points, there is cracking around a fruit umbilicus: 3 points, there is cracking at a fruit umbilicus: 2 points, there is cracking at a fruit pedicel: 1 point, there is a wound on a fruit: 0 point, and there is cracking around a needle: 0 point;
the injected water volume at fruit turgor pressure limit is an amount of injected water when a citrus fruit cracks;
a determination method of the cracked fruit proportion index includes: injecting water into fruits from fruit pedicels of the fruits in an amount 5% to 15% of a weight of each fruit, taking a ratio of the number of fruits in which a peel cracks within 24 h to the total number of fruits in which the water is injected as a cracking ratio Cr after the water injection, and calculating the cracked fruit proportion index with the cracking ratio Cr after the water injection, a score S of a peel cracking site, and a time t when a peel cracks according to a predetermined formula:
cracked fruit proportion index=0.347×(25−t)×(1+S)×(1+Cr), where the score S of a peel cracking site is determined by the same scoring manner as the fruit cracking site at fruit turgor pressure limit, and the time t when a peel cracks refers to the time of hour t after the water is injected into a fruit;
the peel hardness, the fruit turgor pressure, and the injected water volume at fruit turgor pressure limit each are negatively correlated with a fruit cracking rate of citrus fruits; and
the fruit cracking site at fruit turgor pressure limit and the cracked fruit proportion index each are positively correlated with a fruit cracking rate of citrus fruits.

Preferably, a determination method of the fruit turgor pressure includes: connecting one end of a water injection tube to a syringe needle, and connecting the other end of the water injection tube to a tip of the syringe, with a water pressure gauge connected between the syringe needle and the tip of the syringe; inserting the syringe needle into a citrus fruit until the syringe needle reaches a central axis of the citrus fruit, and injecting water into the citrus fruit by the syringe; and taking a pressure value displayed by the water pressure gauge when the citrus fruit cracks as the fruit turgor pressure.

The present disclosure also provides the use of the method in the above technical solution in breeding and/or cultivation of a citrus.

Preferably, the cultivation includes citrus fruit cracking control and/or water and fertilizer management.

### Beneficial Effects

The present disclosure provides the use of peel hardness, fruit turgor pressure, fruit cracking site at fruit turgor pressure limit, injected water volume at fruit turgor pressure limit, and cracked fruit proportion index in evaluation of citrus fruit cracking. In the present disclosure, five indicators for evaluating citrus fruit cracking are determined and subjected to correlation analysis with field experiment data of fruit cracking rates of citrus fruits, and analysis results show that these five indicators are positively correlated with a cracking rate of fruits. By detecting these five indicators, the correlation of fruit cracking characteristics with a variety and cultivation conditions can be accurately described to fundamentally understand a cause for fruit cracking. In addition, it is convenient to determine the five indicators, and the five indicators can be detected in both fruits on a fruit tree and picked fruits, which help to reflect a fruit cracking situation of fruits and find a measure for accurately reducing the fruit cracking. The present disclosure provides indicators of detection and/or reference for improving the fruit cracking characteristics of a variety through breeding or guiding measures for controlling the fruit cracking including application of a specified fertilizer or reduction of a watering quantity.

### DETAILED DESCRIPTION OF THE EMBODIMENTS

The present disclosure provides a use of one or more of five indicators in evaluation of citrus fruit cracking.

The five indicators include peel hardness, fruit turgor pressure, fruit cracking site at fruit turgor pressure limit, injected water volume at fruit turgor pressure limit, and cracked fruit proportion index.

The fruit turgor pressure is a pressure value produced inside a citrus fruit when the citrus fruit cracks due to water injected into the citrus fruit from a central axis of the citrus fruit.

The fruit cracking site at fruit turgor pressure limit is a site of a crack of a citrus fruit, and the fruit cracking site at fruit turgor pressure limit is quantified by the following scoring manner: there is cracking in a peel equatorial plane: 5 points, there is cracking between a fruit equatorial plane and a fruit pedicel or a fruit umbilicus: 4 points, there is cracking around a fruit umbilicus: 3 points, there is cracking at a fruit umbilicus: 2 points, there is cracking at a fruit pedicel: 1 point, there is a wound on a fruit: 0 point, and there is cracking around a needle: 0 point. In the present disclosure, the wound on a fruit is preferably caused by a needle or the like during water injection; and the cracking is preferably fruit cracking caused by a fruit turgor pressure, and can simulate a process of actual fruit cracking. Further, the wound and the cracking are obviously different from each other, and can be routinely identified by those skilled in the art.

The injected water volume at fruit turgor pressure limit is an amount of injected water when a citrus fruit cracks.

A determination method of the cracked fruit proportion index includes: water is injected into fruits from fruit pedicels of the fruits in an amount 5% to 15% of a weight of each fruit, a ratio of the number of fruits in which a peel cracks within 24 h to the total number of fruits in which the water is injected is taken as a cracking ratio Cr after the water injection, and the cracked fruit proportion index is calculated with the cracking ratio Cr after the water injection, a score S of a peel cracking site, and a time t when a peel cracks according to a predetermined formula:

cracked fruit proportion index=0.347×(25−t)×(1+S)×(1+Cr), where the score S of a peel cracking site is determined by the same scoring manner as the fruit cracking site at fruit turgor pressure limit, and the time t when a peel cracks refers to a tₜₕ h after the water is injected into a fruit.

In the present disclosure, the peel hardness preferably includes peel hardness of an intact fruit and isolated peel hardness. In the present disclosure, the peel hardness is determined preferably by a texture analyzer. The brand and the model of the texture analyzer are not specially limited, and a conventional texture analyzer in the art may be adopted. For example, in an embodiment of the present disclosure, a texture analyzer with a model of TA-XT plus 40738 from the Stable Micro Systems (UK) is adopted. The determination of the peel hardness is conducted with a depth of preferably 3 mm to 8 mm and more preferably 5 mm. The depth for the determination of the peel hardness has advantages such as conforming to thicknesses of most of peels and high data reproducibility. The determination of the peel hardness is conducted with a probe diameter of preferably 2 mm and a speed of preferably 1.0 mm/s. In the present disclosure, when the peel hardness of an intact fruit is measured, two symmetry points of an equatorial plane are preferably selected for detection, and then an average value is taken as the peel hardness of an intact fruit obtained after one measurement. When the isolated peel hardness is measured, a square peel tissue with a side length of 1 cm to 3 cm is preferably selected for detection, and a square peel tissue with a side length of 2 cm is more preferably selected for detection, where a white side of the square peel tissue faces upwards.

In the present disclosure, a determination method of the fruit turgor pressure preferably includes: one end of a water injection tube is connected to a syringe needle, and the other end of the water injection tube is connected to the tip of the syringe, with a water pressure gauge connected between the syringe needle and the tip of the syringe; the syringe needle is inserted into a citrus fruit until the syringe needle reaches a central axis of the citrus fruit, and water is injected into the citrus fruit by the syringe; and a pressure value displayed by the water pressure gauge when the citrus fruit cracks is taken as the fruit turgor pressure.

The amount of injected water when a citrus fruit cracks in the present disclosure is an injected water volume at fruit turgor pressure limit.

In the present disclosure, a determination method of the cracked fruit proportion index includes: water is injected into fruits from fruit pedicels of the fruits in an amount 5% to 15% of the weight of each fruit, a ratio of the number of fruits in which a peel cracks within 24 h to the total number of fruits in which the water is injected is taken as a cracking ratio Cr after the water injection, and the cracked fruit proportion index is calculated with the cracking ratio Cr after the water injection, a score S of a peel cracking site, and a time t when a peel cracks according to a predetermined formula:
cracked fruit proportion index=0.347×(25−t)×(1+S)×(1+Cr), where the score S of a peel cracking site is determined by the same scoring manner as the fruit cracking site at fruit turgor pressure limit.

In the present disclosure, a weight of the water is preferably 10% of the weight of a fruit. The time t when a peel cracks in the present disclosure is preferably an integer value in a range of 1 to 24, and specific scoring rules are explained as follows: there is cracking at 0 h to 1 h: 1 point, there is cracking at 1 h to 2 h: 2 points, there is cracking at 2 h to 3 h: 3 points, there is cracking at 3 h to 4 h: 4 points . . . there is cracking at 22 h to 23 h: 23 points, there is cracking at 23 h to 24 h: 24 points, and there is cracking after more than 24 h or there is no cracking: 24 points.

In the present disclosure, the peel hardness is preferably negatively correlated with the fruit cracking rate of citrus fruits, that is, the higher the peel hardness is, the lower the fruit cracking rate of citrus fruits is. The fruit turgor pressure is preferably negatively correlated with a fruit cracking rate of citrus fruits, that is, the higher the fruit turgor pressure is, the lower the fruit cracking rate of citrus fruits is. The injected water volume at fruit turgor pressure limit is preferably negatively correlated with a fruit cracking rate of citrus fruits, that is, the larger the injected water volume at fruit turgor pressure limit is, the lower the fruit cracking rate of citrus fruits is. The fruit cracking site at fruit turgor pressure limit is preferably positively correlated with a fruit cracking rate of citrus fruits, that is, the higher the score of the fruit cracking site at fruit turgor pressure limit is, the higher the fruit cracking rate of citrus fruits is. The cracked fruit proportion index is preferably positively correlated with a fruit cracking rate of citrus fruits, that is, the higher the cracked fruit proportion index, the higher the fruit cracking rate of citrus fruits.

The present disclosure also provides a method for evaluating citrus fruit cracking, including: fruit cracking of a citrus fruit is evaluated by determining one or more of five indicators,
where the five indicators include peel hardness, fruit turgor pressure, fruit cracking site at fruit turgor pressure limit, injected water volume at fruit turgor pressure limit, and cracked fruit proportion index;
the fruit turgor pressure is a pressure value produced inside a citrus fruit when the citrus fruit cracks due to water injected into the citrus fruit from a central axis of the citrus fruit;
the fruit cracking site at fruit turgor pressure limit is a site of a crack of a citrus fruit, and the fruit cracking site at fruit turgor pressure limit is quantified by the following scoring manner: there is cracking in a peel equatorial plane: 5 points, there is cracking between a fruit equatorial plane and a fruit pedicel or a fruit umbilicus: 4 points, there is cracking around a fruit umbilicus: 3 points, there is cracking at a fruit umbilicus: 2 points, there is cracking at a fruit pedicel: 1 point, there is a wound on a fruit: 0 point, and there is cracking around a needle: 0 point;
the injected water volume at fruit turgor pressure limit is an amount of injected water when a citrus fruit cracks;
a determination method of the cracked fruit proportion index includes: water is injected into fruits from fruit pedicels of the fruits in an amount 5% to 15% of the weight of each fruit, a ratio of the number of fruits in which a peel cracks within 24 h to the total number of fruits in which the water is injected is taken as a cracking ratio Cr after the water injection, and the cracked fruit proportion index is calculated with the cracking ratio Cr after the water injection, a score S of a peel cracking site, and a time t when a peel cracks according to a predetermined formula:
cracked fruit proportion index=0.347×(25−t)×(1+S)×(1+Cr), where the score S of a peel cracking site is determined by the same scoring manner as the fruit cracking site at fruit turgor pressure limit, and the time t when a peel cracks refers to the time of hour t after the water is injected into a fruit;
the peel hardness, the fruit turgor pressure, and the injected water volume at fruit turgor pressure limit each are negatively correlated with a fruit cracking rate of citrus fruits; and
the fruit cracking site at fruit turgor pressure limit and the cracked fruit proportion index each are positively correlated with a fruit cracking rate of citrus fruits.

The processes and parameters for determining the five indicators of the present disclosure have been optimized and defined in the above-mentioned technical solutions and are not repeated herein.

In the present disclosure, the higher the peel hardness is, the lower the fruit cracking rate of citrus fruits; the larger the injected water volume at fruit turgor pressure limit is, the lower the fruit cracking rate of citrus fruits is; the higher the score of the fruit cracking site at fruit turgor pressure limit is, the higher the fruit cracking rate of citrus fruits is; and the higher the cracked fruit proportion index is, the higher the fruit cracking rate of citrus fruits is.

In the present disclosure, when the citrus fruit cracking is evaluated by the method, it is preferable to select a statistically-significant number of fruit trees for determination according to an actual size of an orchard. Among the five indicators, a group of citrus fruits are preferably adopted for determination of the peel hardness; a group of fruits are preferably adopted for determination of the fruit turgor pressure, the fruit cracking site at fruit turgor pressure limit, and the injected water volume at fruit turgor pressure limit; and a group of fruits are preferably adopted for determination of the cracked fruit proportion index, where a statistically-significant number of biological replicates are preferably set for each group of fruits.

In the present disclosure, five indicators for evaluating citrus fruit cracking are determined and subjected to correlation analysis with field experiment data of fruit cracking rates of citrus fruits, and analysis results show that these five indicators are positively correlated with a cracking rate of fruits. By detecting these five indicators, the correlation of fruit cracking characteristics with a variety and cultivation conditions can be accurately described to fundamentally understand a cause for fruit cracking. In addition, it is convenient to determine the five indicators, and the five indicators can be detected in both fruits on a fruit tree and picked fruits, which helps to reflect a fruit cracking situation of fruits and find a measure for accurately reducing the fruit cracking. The present disclosure provides a technical support for improving the fruit cracking characteristics of a variety through breeding or guiding measures for controlling the fruit cracking including application of a specified fertilizer or reduction of a watering quantity.

Based on the above-mentioned advantages, the present disclosure also provides the use of the method described in the above-mentioned technical solution in breeding and/or cultivation of a citrus, and preferably a use of the method in breeding and cultivation of the citrus. The cultivation of the present disclosure preferably includes citrus fruit cracking control and/or water and fertilizer management, and more preferably includes the citrus fruit cracking control and the water and fertilizer management.

In order to further illustrate the present disclosure, the technical solutions provided by the present disclosure are described in detail below in connection with examples, but these examples should not be construed as limiting the claimed scope of the present disclosure.

### Example 1

The fruit cracking of different fruit trees of the citrus variety “Ganping” with a high cracked fruit proportion was evaluated, including the following steps:

In the “Ganping” orchard in Xiangshan County, Ningbo, Zhejiang Province, 10 fruit trees (a total of 200 fruit trees in the orchard) were randomly selected, and fruit cracking rates of these 10 fruit trees in August were recorded (50 fruits were counted per fruit tree of the 10 fruit trees, and an average fruit cracking rate of each fruit tree was taken).

30 citrus fruits were randomly selected from each fruit tree as an evaluation group. The fruit turgor pressure, the fruit cracking site at fruit turgor pressure limit, the injected water volume at fruit turgor pressure limit, and the cracked fruit proportion index after injection of a specified amount of water into fruits were detected in the orchard, and the peel hardness was detected in the laboratory. The fruit turgor pressure, the fruit cracking site at fruit turgor pressure limit, and the injected water volume at fruit turgor pressure limit were detected with the same 10 citrus fruits, the peel hardness was detected with 10 citrus fruits, and the cracked fruit proportion index after injection of a specified amount of water into fruits was detected with 10 bacilli.

### Detection Method:

### (1) Peel Hardness Including Peel Hardness of an Intact Fruit and Isolated Peel Hardness

Peel hardness of an intact fruit: The peel hardness of a citrus fruit was determined by a texture analyzer (TA-XT plus 40738, Stable Micro Systems, UK) with a probe diameter of 2 mm, a depth of 5 mm, and a speed of 1.0 mm/s. 10 fruits were selected from each citrus variety, and two symmetry points on an equatorial plane were selected for detection.

Isolated peel hardness: A peel was carefully peeled off, and 2 cm-square peel tissues were collected, placed on a same rigid plastic plate with a white peel layer facing upwards, and tested by a same texture analyzer for peel hardness with a probe diameter of 2 mm, a depth of 5 mm, and a speed of 1.0 mm/s.

10 fruits were tested per fruit tree, and the peel hardness of an intact fruit and the isolated peel hardness were determined on a same fruit.

### (2) Fruit Turgor Pressure

The fruit turgor pressure was detected by a water injection method. One end of a hose was connected to a syringe needle and a water pressure gauge, and the other end of the horse was connected to the tip of a 50 mL syringe. During a test, the needle was inserted by a pressing machine into a citrus fruit until the needle was close to a fruit pedicel and reached a central axis of the citrus fruit, and water was injected by the syringe into the citrus fruit. A reaction force after water injection created a pressure produced inside the citrus fruit, and this pressure could be recorded by the water pressure gauge. When the amount of injected water reached a maximum value and the citrus fruit cracked, the maximum pressure value displayed by the water pressure gauge was a maximum turgor pressure value that could be withstood by the citrus fruit.

### (3) Fruit Cracking Site at Fruit Turgor Pressure Limit

When a fruit turgor pressure reached a maximum value, a peel cracked and the fruit turgor pressure dropped sharply, at which point a peel cracking site is the fruit cracking site at fruit turgor pressure limit. There was cracking in a peel equatorial plane: 5 points, there was cracking between a fruit equatorial plane and a fruit pedicel or a fruit umbilicus: 4 points, there was cracking around a fruit umbilicus: 3 points, there was cracking at a fruit umbilicus: 2 points, there is cracking at a fruit pedicel: 1 point, there was a wound on a fruit: 0 point, and there was cracking around a needle: 0 point.

### (4) Injected Water Volume at Fruit Turgor Pressure Limit

The volume of injected water required for the fruit turgor pressure to reach the maximum value and the peel to crack was the injected water volume when there was a limit fruit turgor pressure.

(5) Cracking Ratio after Injection of a Specified Amount of Water into Fruits

Water was injected into fruits from fruit pedicels of the fruits in an amount 10% of the weight of each fruit, and the ratio of the number of fruits in which the peel cracked within 24 h to the total number of fruits in which the water was injected was the cracking ratio after injection of a specified amount of water into fruits. Proportion index calculation formula: Pi=0.347×(25−t)×(1+S)×(1+Cr), where t represents a time when a peel cracks, S represents a score of a peel cracking site (where the scoring of a peel cracking site is the same as in the step 3), and Cr represents a cracking ratio.

Results are shown in Tables 1 and 2:

TABLE 1
Indicators for evaluation of cracking of the citrus fruit “Ganping”

| | Average | Average | Fruit | Average | Injected water | Cracked | |
|---|---|---|---|---|---|---|---|
| Fruit | hardness | hardness of | turgor | fruit | volume at turgor | fruit | Fruit |
| tree | of intact | isolated | pressure | cracking | pressure limit | proportion | cracking |
| No. | peel(N) | peel (N) | (kPa) | site | (mL) | index | rate |
| 1 | 10.04 | 2.32 | 34.94 | 4.45 | 32.37 | 90.71 | 0.72 |
| 2 | 8.04 | 2.25 | 36.91 | 3.84 | 26.84 | 80.66 | 0.74 |
| 3 | 11.03 | 2.74 | 51.41 | 3.06 | 39.32 | 64.32 | 0.58 |
| 4 | 7.00 | 1.07 | 18.51 | 4.78 | 12.80 | 96.26 | 0.84 |
| 5 | 10.54 | 2.24 | 43.77 | 4.16 | 32.04 | 81.71 | 0.70 |
| 6 | 7.26 | 1.18 | 20.81 | 4.36 | 15.20 | 89.24 | 0.82 |
| 7 | 6.30 | 1.95 | 36.28 | 3.90 | 37.74 | 77.51 | 0.76 |
| 8 | 9.92 | 2.85 | 48.55 | 3.56 | 44.89 | 75.92 | 0.64 |
| 9 | 14.30 | 2.80 | 52.38 | 2.99 | 42.50 | 66.45 | 0.54 |
| 10 | 13.68 | 2.85 | 59.14 | 3.32 | 43.37 | 71.94 | 0.58 |

TABLE 2
Correlation analysis between fruit cracking indicators and the
fruit cracking rate of the citrus fruit “Ganping”

| Fruit cracking indicator | Correlation (r²) |
|---|---|
| Average intact peel hardness | 0.82 |
| Average isolated peel hardness | 0.85 |
| Fruit turgor pressure | 0.90 |
| Average fruit cracking site | 0.84 |
| Injected water volume at turgor pressure limit | 0.75 |
| Proportion index | 0.79 |

As shown in Table 1, the fruit cracking of Ganping fruits was evaluated with the above indicators, and the peel hardness of an intact fruit and the isolated peel hardness were detected in the laboratory. The average intact peel hardness of 10 fruit trees was 6.30 N to 14.30 N, and the average isolated peel hardness of the 10 fruit trees was 1.07 N to 2.85 N. The intact peel hardness and the isolated peel hardness were negatively correlated with the fruit cracking rate of a corresponding fruit tree, with correlation coefficients of 0.82 and 0.85, respectively. The fruit turgor pressure, the average fruit cracking site, the injected water volume at turgor pressure limit, and the cracked fruit proportion index when a specified volume of water is injected were directly detected in fruits on a fruit tree. The fruit turgor pressure directly reflects a maximum internal pressure that could be withstood by a peel, and was 18.51 kPa to 59.14 kPa. The fruit cracking site determined the vulnerable part inside the peel. Most of Ganping fruits underwent equatorial plane cracking, and a corresponding score was 2.99 to 4.78. The injected water volume at maximum turgor pressure determined a maximum water volume that could be withstood inside a fruit and was 12.80 mL to 44.89 mL. After water was injected into the fruit in an amount 10% of the weight of the fruit, most of Ganping fruits crack within 1 h, the proportion of cracked fruits reached 90% or more, and the cracked fruit proportion index was 64.32 to 96.26.

The average intact peel hardness, the average isolated peel hardness, the fruit turgor pressure, the average fruit cracking site, the injected water volume at turgor pressure limit, and the proportion index determined in Table 1 each were subjected to correlation analysis with a fruit cracking rate. A specific method was as follows: a dot diagram was plotted with a fruit cracking indicator as an x-coordinate and a fruit cracking rate as a y-coordinate, then a trend line was added, and an R2 value was displayed. The larger the R2 value, the higher the correlation between a fruit indicator and a fruit cracking rate. Results are shown in Table 2. The correlation analysis results show that the fruit turgor pressure and the injected water volume when there is a limit turgor pressure each are negatively correlated with a fruit cracking rate of fruits, with correlation coefficients of 0.90 and 0.75, respectively, and the average fruit cracking site and the cracked fruit proportion index each are positively correlated with a fruit cracking rate, with correlation coefficients of 0.84 and 0.79, respectively.

The above results show that the indicators such as the peel hardness, the fruit turgor pressure, the average fruit cracking site, the injected water volume when there is a limit turgor pressure, and the cracked fruit proportion index can reflect a fruit cracking rate level of fruits, and allow a fruit cracking rate to be quantified.

### Example 2

The fruit cracking of “Ganping” citrus fruits picked from different fruit trees in a same orchard was evaluated, including the following steps:

In the “Ganping” orchard in Quzhou City, Zhejiang Province, 10 fruit trees were randomly selected, and fruit cracking rates of these 10 fruit trees in August were recorded (50 fruits were counted per fruit tree of the 10 fruit trees, and an average fruit cracking rate of each fruit tree was taken).

30 citrus fruits were randomly selected from each fruit tree as an evaluation group. The peel hardness, the fruit turgor pressure, the fruit cracking site when the fruit turgor pressure reaches a limit, the injected water volume at fruit turgor pressure limit, and the cracked fruit proportion index after injection of a specified amount of water into fruits each were detected in the laboratory.

A detection method is shown in Example 1. Results are shown in Tables 3 and 4:

TABLE 3
Indicators for evaluation of cracking of the citrus fruit “Ganping”

| | Average | Average | Fruit | Average | Injected water | Cracked | |
|---|---|---|---|---|---|---|---|
| Fruit | hardness of | hardness of | turgor | fruit | volume at turgor | fruit | Fruit |
| tree | intact peel | isolated | pressure | cracking | pressure limit | proportion | cracking |
| No. | (N) | peel (N) | (kPa) | site | (mL) | index | rate |
| 1 | 11.40 | 2.79 | 60.48 | 3.22 | 47.75 | 70.35 | 0.58 |
| 2 | 12.44 | 3.19 | 59.19 | 3.02 | 48.33 | 66.99 | 0.56 |
| 3 | 8.34 | 2.08 | 29.33 | 4.96 | 21.66 | 94.26 | 0.76 |
| 4 | 8.38 | 2.35 | 40.09 | 4.30 | 34.88 | 88.27 | 0.68 |
| 5 | 8.45 | 2.12 | 41.54 | 4.71 | 34.50 | 90.42 | 0.70 |
| 6 | 8.30 | 1.73 | 35.63 | 4.69 | 21.88 | 94.77 | 0.76 |
| 7 | 6.67 | 1.78 | 20.17 | 4.97 | 24.70 | 94.53 | 0.82 |
| 8 | 10.60 | 2.29 | 54.61 | 4.60 | 40.25 | 88.67 | 0.66 |
| 9 | 11.13 | 2.36 | 44.92 | 3.94 | 36.94 | 69.94 | 0.62 |
| 10 | 10.98 | 1.90 | 38.75 | 3.99 | 31.25 | 83.14 | 0.70 |

TABLE 4
Correlation analysis between fruit cracking indicators and the
fruit cracking rate of the citrus fruit “Ganping”

| | Correlation |
|---|---|
| Fruit cracking indicator | (r²) |
| Average intact peel hardness | 0.81 |
| Average isolated peel hardness | 0.82 |
| Fruit turgor pressure | 0.88 |
| Average fruit cracking site | 0.80 |
| Injected water volume when there is a limit turgor pressure | 0.88 |
| Proportion index | 0.82 |

As shown in Table 3, the fruit cracking of picked Ganping fruits was evaluated with the indicators shown in Table 3, and all indicators were detected in the laboratory. The results show that an average intact peel hardness of the picked Ganping fruits was 6.67 N to 12.44 N, and an average isolated peel hardness of the picked Ganping fruits was 1.73 N to 3.19 N. The intact peel hardness and the isolated peel hardness were negatively correlated with the fruit cracking rate of a corresponding fruit tree, with correlation coefficients of 0.81 and 0.82, respectively. The fruit turgor pressure was in a range of 20.17 kPa to 60.48 kPa. The fruit cracking site determined the vulnerable part inside the peel. Most of Ganping fruits undergo equatorial plane cracking, and a corresponding score was 3.02 to 4.97. The injected water volume at maximum turgor pressure determined a maximum water volume that could be withstood inside a fruit, and is 21.66 mL to 48.33 mL. After water was injected into a fruit in an amount 10% of a weight of the fruit, most of Ganping fruits crack within 1 h, a proportion of cracked fruits reached 90% or more, and a cracked fruit proportion index is 66.99 to 94.77.

As shown by the results of correlation analysis between the indicators and a fruit cracking rate in Table 4, the fruit turgor pressure and the injected water volume at limit turgor pressure each are negatively correlated with a fruit cracking rate of fruits, with correlation coefficients both of 0.88, and the average fruit cracking site and the cracked fruit proportion index each are positively correlated with a fruit cracking rate, with correlation coefficients of 0.80 and 0.82, respectively.

The above results show that the indicators such as the peel hardness, the fruit turgor pressure, the average fruit cracking site, the injected water volume when there is a limit turgor pressure, and the cracked fruit proportion index in the method can also accurately reflect a fruit cracking rate level of picked fruits, and allow a fruit cracking rate to be quantified.

### Example 3

The fruit cracking of different citrus varieties was evaluated, including the following steps:

In the orchards of various citrus varieties in Taizhou City, Zhejiang Province, 10 citrus varieties were selected: “Ganping”, “Hong Mei Ren”, “Ming Ri Jian”, “Hongying”, “Eureka”, “Fertile Orange”, “Chunjian”, “Moyang Ponkan”, “Zijin Wenchow Orange”, and “Chachiensis”. 10 fruit trees were randomly selected from each variety, and fruit cracking rates of these 10 fruit trees in August were recorded (30 fruits were counted per fruit tree of the 50 fruit trees, and an average fruit cracking rate of each fruit tree was taken).

30 citrus fruits were randomly selected from each fruit tree as an evaluation group. The peel hardness, the fruit turgor pressure, the fruit cracking site at fruit turgor pressure limit, the injected water volume at fruit turgor pressure limit, and the cracked fruit proportion index after injection of a specified amount of water into fruits each were detected in the laboratory. The peel hardness was detected in the laboratory.

A detection method is shown in Example 1. Results are shown in Tables 5 and 6:

TABLE 5
Peel hardnesses of fruits of 10 citrus varieties

| | Average | Average | Fruit | Average | Injected water | Cracked | |
|---|---|---|---|---|---|---|---|
| | hardness of | hardness of | turgor | fruit | volume at turgor | fruit | Fruit |
| Citrus | intact peel | isolated peel | pressure | cracking | pressure limit | proportion | cracking |
| variety | (N) | (N) | (kPa) | site | (mL) | index | rate |
| Ganping | 10.45 | 2.46 | 39.78 | 4.80 | 26.30 | 96.60 | 0.68 |
| Hong Mei | 21.58 | 5.05 | 84.49 | 2.51 | 30.90 | 58.52 | 0.23 |
| Ren | | | | | | | |
| Ming Ri | 13.89 | 3.03 | 70.24 | 4.01 | 20.91 | 79.28 | 0.54 |
| Jian | | | | | | | |
| Hongying | 21.68 | 5.12 | 85.68 | 2.39 | 38.09 | 56.46 | 0.08 |
| Eureka | 21.83 | 5.35 | 99.50 | 0.51 | 43.81 | 23.83 | 0.02 |
| Fertile | 24.37 | 5.02 | 114.31 | 0.74 | 42.05 | 28.97 | 0.02 |
| Orange | | | | | | | |
| Chunjian | 26.02 | 5.24 | 90.23 | 0.59 | 40.39 | 25.17 | 0.08 |
| Moyang | 22.55 | 5.01 | 104.54 | 0.50 | 42.39 | 23.77 | 0.02 |
| Ponkan | | | | | | | |
| Zijin | 22.37 | 4.58 | 106.15 | 0.25 | 41.51 | 17.66 | 0.00 |
| Wenchow | | | | | | | |
| Orange | | | | | | | |
| Chachiensis | 22.02 | 5.21 | 94.80 | 0.58 | 38.59 | 26.34 | 0.04 |

TABLE 6
Correlation analysis between fruit cracking indicators and
fruit cracking rates of fruits of the 10 citrus varieties

| | Correlation |
|---|---|
| Fruit cracking indicator | (r²) |
| Average intact peel hardness | 0.87 |
| Average isolated peel hardness | 0.87 |
| Fruit turgor pressure | 0.86 |
| Average fruit cracking site | 0.90 |
| Injected water volume when there is a limit turgor pressure | 0.86 |
| Proportion index | 0.89 |

As shown in Table 5, the fruit cracking rates and fruit cracking-associated indicators of the 10 different citrus varieties were evaluated. The results show that the average intact peel hardness of picked citrus fruits was 10.45 N to 26.02 N, where the “Ganping” and “Ming Ri Jian” with relatively-high fruit cracking rates had the lowest intact peel hardness, which were 10.45 N and 13.89 N, respectively. The average isolated peel hardness was 2.46 N to 5.35 N, where the “Ganping” and “Ming Ri Jian” with relatively-high fruit cracking rates also had the lowest isolated peel hardness. The intact peel hardness and the isolated peel hardness were negatively correlated with a fruit cracking rate of a corresponding fruit tree, with correlation coefficients both of 0.87. The fruit turgor pressure was 39.78 kPa to 114.31 kPa, where the “Ganping” and “Ming Ri Jian” had relatively-low fruit turgor pressures, which were are 39.78 kPa and 70.24 kPa, respectively. The fruit cracking site determined a vulnerable part inside a peel, and a corresponding score was 0.25 to 4.80, where cracking mainly occured around a needle or a fruit pedicel in the “Zijin Wenchow Orange”, “Chachiensis”, “Moyang Ponkan”, and “Eureka” with relatively-low fruit cracking rates, and an index was low. The injected water volume at maximum turgor pressure determined a maximum water volume that could be withstood inside the fruit, and was 20.97 mL to 43.81 mL. After water was injected into a fruit in an amount 10% of the weight of the fruit, the cracked fruit proportion index was 17.66 to 96.60, where a cracked fruit proportion index of the “Ganping” was 96.60, and cracked fruit proportion indexes of the “Zijin Wenchow Orange”, “Chachiensis”, and “Eureka” with relatively-low fruit cracking rates were 30% or less.

As shown by the results of correlation analysis between the indicators and a fruit cracking rate in Table 6, the fruit turgor pressure and the injected water volume at turgor pressure limit each are negatively correlated with a fruit cracking rate of fruits, with correlation coefficients both of 0.86, and the average fruit cracking site and the cracked fruit proportion index each are positively correlated with a fruit cracking rate, with correlation coefficients of 0.90 and 0.89, respectively.

The above results show that the indicators such as the peel hardness, the fruit turgor pressure, the average fruit cracking site, the injected water volume when there is a limit turgor pressure, and the cracked fruit proportion index in the method of the present disclosure can also accurately reflect fruit cracking rate levels of fruits of different citrus varieties, and allow a fruit cracking rate to be quantified.

Although the present disclosure has been described in detail through the above examples, the examples are merely some rather than all of the examples of the present disclosure. All other examples obtained by a person based on these examples without creative efforts shall fall within the protection scope of the present disclosure.

What is claimed is:

1. A method for evaluating citrus fruit cracking, comprising: evaluating fruit cracking of a citrus fruit by determining one or more of five indicators,
wherein the five indicators comprise peel hardness, fruit turgor pressure, fruit cracking site at fruit turgor pressure limit, injected water volume at fruit turgor pressure limit, and cracked fruit proportion index;
the fruit turgor pressure is a pressure value produced inside a citrus fruit when the citrus fruit cracks due to water injected into the citrus fruit from a central axis of the citrus fruit;
the fruit cracking site at fruit turgor pressure limit is a site of a crack of a citrus fruit, and the fruit cracking site at fruit turgor pressure limit is quantified by the following scoring manner: there is cracking in a peel equatorial plane: 5 points, there is cracking between a fruit equatorial plane and a fruit pedicel or a fruit umbilicus: 4 points, there is cracking around a fruit umbilicus: 3 points, there is cracking at a fruit umbilicus: 2 points, there is cracking at a fruit pedicel: 1 point, there is a wound on a fruit: 0 point, and there is cracking around a needle: 0 point;
the injected water volume at fruit turgor pressure limit is an amount of injected water when a citrus fruit cracks;
a determination method of the cracked fruit proportion index comprises: injecting water into fruits from fruit pedicels of the fruits in an amount 5% to 15% of a weight of each fruit, taking a ratio of the number of fruits in which a peel cracks within 24 h to the total number of fruits in which the water is injected as a cracking ratio Cr after the water injection, and calculating the cracked fruit proportion index with the cracking ratio Cr after the water injection, a score S of a peel cracking site, and a time t when a peel cracks according to a predetermined formula:
cracked fruit proportion index=0.347×(25−t)×(1+S)×(1+Cr), wherein the score S of a peel cracking site is determined by the same scoring manner as the fruit cracking site at fruit turgor pressure limit, and the time t when a peel cracks refers to a tₜₕ h after the water is injected into a fruit;
the peel hardness, the fruit turgor pressure, and the injected water volume at fruit turgor pressure limit each are negatively correlated with a fruit cracking rate of citrus fruits; and
the fruit cracking site at fruit turgor pressure limit and the cracked fruit proportion index each are positively correlated with a fruit cracking rate of citrus fruits.

2. The method according to claim 1, wherein a determination method of the fruit turgor pressure comprises: connecting one end of a water injection tube to a syringe needle, and connecting the other end of the water injection tube to a tip of the syringe, with a water pressure gauge connected between the syringe needle and the tip of the syringe; inserting the syringe needle into a citrus fruit until the syringe needle reaches a central axis of the citrus fruit, and injecting water into the citrus fruit by the syringe; and taking a pressure value displayed by the water pressure gauge when the citrus fruit cracks as the fruit turgor pressure.

3. The method according to claim 1, wherein the peel hardness comprises a peel hardness of an intact fruit and hardness of an isolated peel; and the hardness of the peel is determined by a texture analyzer with a depth of 3 mm to 8 mm.

4. The method according to claim 2, wherein the texture analyzer has a probe diameter of 2 mm and a travel speed of 1.0 mm/s.
